(19) United States
(12) Patent Application Publication
(10) Pub. No.: US 2006/0013864 A1
(43) Pub. Date: Jan. 19, 2006

(54) Transmucosal pharmacuetical administration form

(76) Inventors: Hans-Rainer Hoffmann, Neuwied (DE); Reinhard von Kleinsorgen, Bendorf (DE); Werner Wessling Beethovenstr, Rengsdorf (DE)

Correspondence Address: William F Lawrence;Frommer Lawrence & Haug, 745 Fifth Avenue, New York, NY, 10151, US

(21) Appl. No.: 10/533,926

(22) PCT Filed: Oct. 17, 2003
(86) PCT No.: PCT/EP03/11529

§ 371 Date: May 4, 2005

(30) Foreign Application Priority Data
Nov. 8, 2002 (DE) 102 51 963.3

(51) Int. Cl. A61K 31/403; A61L 15/16

(52) U.S. Cl. 424/448; 514/412

(57) ABSTRACT

Planiform transmucosal pharmaceutical administration forms are disclosed, comprising a solid solution of the active ingredient in a phosphatidyl fraction, or a mixture of said phosphatidyl fraction with a copolymer of maleic acid and an alkyl vinyl ether. The administration forms are characterised by a low solubility in the buccal cavity, which gives a rapid and constant active ingredient release over an extended period. The above are particularly suitable for the treatment of the abuse of and dependence on addictive drugs.

The invention relates to administration forms which are preferably planiform and which form liquid-crystalline structures or phases in an aqueous environment, in particular to oral administration forms which can be used to permit controlled absorption of active compounds in the oral cavity, in particular in the unkeratinized regions, and which possess a matrix which is based on phospholipids as basic substances. In particular, the invention relates to administration forms of said type which are configured in the form of wafers. The invention also encompasses a process for producing these administration forms.

The invention enables a wide spectrum of active compounds, e.g. active compounds which act in the CNS (central nervous system), in the cardiovascular system, in the muscle and skeletal system and in the respiratory system of the human body, and also active compounds which act as antiinfective agents, as antibiotics and as hormones, to be delivered in a controlled manner to the oral mucosa.

Preferred active compounds which come into consideration for the administration form according to the invention are those which are suitable for treating drug abuse or drug dependence, in particular for treating nicotine dependence and alcohol dependence of differing genesis. The following substances or substance classes are particularly suitable for this indication: 7-azabicyclo(2.2.1)heptane and -heptene and their derivatives; ebibatidine and derivatives; fused indole derivatives; benzylidene and cinnamylidene-annabasiene; mecamylamine, hypericin, the cannabinoid receptor (CB1) antagonist SR 141716, befloxatone, oxazolidinone derivatives such as pemoline, buproprion and the active compound CP-52655, and also the acid addition salts of the abovementioned substances.

The active compounds, their preparation and their pharmacological effects are described in the following U.S. patent specifications: U.S. Pat. No. 6,255,490; U.S. Pat. No. 6,177,451; U.S. Pat. No. 6,117,889; U.S. Pat. No. 5,998,409 and U.S. Pat. No. 5,977,144.

Pharmaceutical administration forms, e.g. buccal and sublingual tablets, which release active compounds in the oral cavity, with the active compounds then being absorbed through the oral mucosa, are advantageous in a variety of ways. They facilitate the oral administration of medicaments to certain patients who experience difficulty in ingesting other oral medicinal forms, e.g. because of problems with swallowing. Since the absorption takes place through the oral mucosa, and with the gastrointestinal tract being circumvented, rapid onset of effect and high active compound utilization are ensured. In addition to sublingual or buccal tablets, planiform, wafer-like administration forms (also termed wafers) are also suitable for use as oral medicinal forms which exhibit the abovementioned properties. Because of their low layer thickness and their ability to disintegrate, or be dissolved, rapidly, these wafers are particularly suitable for rapidly releasing medicaments and other active compounds in the oral cavity. As a rule, such wafer-like medicinal forms are constructed from film-forming, water-soluble polymers, e.g. particular cellulose derivatives. On contact with water or saliva, the wafer matrix structure, which is formed by the polymers, decomposes, or the structure is dissolved, and the active compounds which are present in it are released. The onset, and the chronological course, of the active compound release depend to a large extent on the thickness of the medicinal form (of the wafer) and on the nature of the matrix structure. The structure of the matrix determines the release (profile); the nature of the polymer, or the nature and composition of the polymer mixture, determines the adherence to the mucosa. Consequently, the thickness of such administration forms is essentially determined by the nature and quantity of the active compound which they contain and are to release. As the thickness increases, the decomposition or dissolution of the wafer is correspondingly retarded. In particular, the relatively thick wafers, but also those having a relatively low thickness, tend, because of their flat, smooth form and the delayed disintegration, to adhere, and stick, to the pallet or to other mucosal surfaces in the oral cavity. This is determined, on the one hand, by the polymer layers which dissolve superficially.

DE-A-100 32 456 and DE-A-101 07 659 describe wafers which have been deliberately configured to exhibit a reduced tendency to adhere or stick to the oral mucosa and to have, as their aim, an accelerated release of the active compound. The dwell time of these administration forms at the site of administration (e.g. the oral cavity), or the disintegration time, is preferably in the range from 5 sec to 1 min, more preferably in the range from 10 sec to 1 min and most preferably in the range from 10 sec to 30 sec. The matrix of these administration forms contains, as basic substances, a water-soluble polymer or mixtures of such polymers. In this connection, preference is given to using synthetic or partially synthetic polymers, or biopolymers of natural origin, which are film-forming and water-soluble and/or which are also suitable, for example, for forming foams.

These documents describe polymers which are preferably selected from the group which comprises cellulose derivatives, polyvinyl alcohol, polyacrylates and polyvinylpyrrolidone as being particularly suitable carrier substances (matrix). The cellulose derivatives which are particularly preferred are hydroxypropylmethyl cellulose, carboxymethyl cellulose, hydroxypropyl cellulose, hydroxymethyl cellulose and methyl cellulose, as well as other substituted cellulose derivatives. Preference is likewise given, in these documents, to water-soluble polysaccharides which are of vegetable, microbial or synthetic origin, in particular polysaccharides which are not cellulose derivatives, for example pullulan, xanthan, alginates, dextrans, agar-agar, pectins and carrageen. Furthermore, proteins, preferably gelatin or other gel-forming proteins, and also protein hydrolysates, are also mentioned. The carrier materials which are suitable in the abovementioned patents or laid-open specifications likewise include caseinates, whey and vegetable proteins, gelatin and (chicken) egg white, and mixtures thereof.

EP-B-0 450 141 discloses a carrier material for administering active compounds, which material is of such a composition that it dissolves rapidly on contact with saliva after having been taken orally. This material is a porous, dehydrated skeleton-like carrier substance which is in particular based on proteins, polysaccharides and/or phorspholipids, such as lecithin, without, however, said lecithin being specified. The gelatin-polysaccharide carriers which are described can also be used in the form of wafers. The carrier substances are at the latest rehydrated on contact with saliva and are thereby given a tacky surface which results in the administration form adhering in the oral cavity.

The wafer systems which are described in said prior art, and their physicochemical construction, suffer from the disadvantage that
1. they dissolve rapidly, which means that any longer-term contact of the active compound with the mucosa, for the purpose of enabling the active compound to be absorbed in the oral region, either does not occur or only occurs to a very limited extent,
2. even if it maintains contact with the mucosa for a relatively long period, the matrix only acts as scaffolding which does not promote penetration.

These properties are disadvantageous for the mucosal administration of active compounds which have to be absorbed rapidly, i.e. which require a rapid onset of effect and which at the same time have to ensure a constant blood level over a relatively long period. These active compounds are, in particular, the above-mentioned substances which are suitable for treating the abuse of addiction-inducing drugs and their dependence on these drugs.

The object of the present invention is therefore to provide a planiform or wafer-like administration system which
1. adheres, for a relatively long period, to the oral mucosa, in particular in the area of the frenulum, of the ventral tongue region or in the floor of the mouth, i.e. the unkeratinized region of the oral cavity,
2. holds the active compound available in a form which permits absorption, in the oral region, which is both rapid and constant over a relatively long period,
3. is tasteless or conveys the sensation of tastelessness.

According to the invention, this object is achieved by the parent substance of the transmucosal administration form being composed of a solid solution of the active compound
a) in a phosphatidylcholine whose fatty acid residues are at least 90% saturated, or
b) in a mixture of the phosphatidylcholine mentioned under a) and a copolymer composed of maleic acid and an alkyl vinyl ether.

The parent substance in accordance with a) and b) can additionally contain further pharmaceutically tolerated adjuvants and additives, for example a polyvinyl-pyrrolidone of medium chain length, with the polyvinylpyrrolidone also serving to improve the taste of the administration form according to the invention.

The phosphatidylcholine fractions Epikuron 180 and/or Epikuron 180H have, in particular, proved suitable for the administration form according to the invention.

When they are dissolved in pure alcohol, it is possible to use these phosphatidylcholines to prepare, by drying, solid transparent films in which the active compound is present as a solid solution. These films adhere to the oral mucosa for a sufficiently long period. When water gains access to these films, myelin-like structures, in which the active compound is still dissolved, issue from the film surface. These structures are not vesicular active compound-“encapsulated” microscopic units but, rather, lamellar mesophases in whose lamellar regions the active compound is present in molecular form. These lamellar mesophases are particularly suitable for becoming attached to the mucosa.

This myelin formation can be controlled, right through to a spontaneously emulsifying gel system similar to a bore oil emulsion, depending on the content of residual solvent (ethanol) or additions of small quantities of pure hydrocarbons (e.g. low-viscosity paraffin) or triglycerides of low hydroxyl number.

1. A planiplaniform transmucosal pharmaceutical administration form which is distinguished by low solubility within the oral cavity and release of active compound which is rapid and constant over a relatively long period, characterized in that it is composed of a solid solution of the active compound
a) in a phosphatidylcholine fraction in which the fatty acid residues are at least 90% saturated, or
b) in a mixture of the phosphatidylcholine fraction specified under a) and a copolymer composed of maleic acid and an alkyl vinyl ether,
and, where appropriate, further pharmaceutically tolerated adjuvants and additives.

2. The administration form as claimed in claim 1, characterized in that it comprises at least 80% by weight of the phosphatidylcholine fraction in accordance with a).

3. The administration form as claimed in claim 1, characterized in that it comprises polyvinylpyrrolidone as additive.

4. The administration form as claimed in claim 1, characterized in that the active compound is suitable for treating the abuse of addiction-inducing drugs and dependence on these drugs.

5. The administration form as claimed in claim 1, characterized in that the active compound is a fused indole derivative and/or its acid addition salt.

6. The administration form as claimed in claim 1, characterized in that the active compound is 7-azabicyclo(2.2.1)heptane, 7-azabicyclo(2.2.1)heptene and/or a derivative of this compound.

7. The administration form as claimed in claim 1, characterized in that the active compound is ebibatidine and/or a derivative of this compound.

8. The administration form as claimed in claim 1, characterized in that the active compound is a benzylidene- and cinnamylidene-annabasiene or a derivative of this compound.

9. The administration form as claimed in claim 1, characterized in that the active compound is selected from the compound group mecamylamine, hypericin, CP-52655 and buproprion and/or one of their derivatives.

10. The administration form as claimed in claim 1, characterized in that the active compound is selected from the group of oxazolidinone derivatives and befloxatones.

11. The administration form as claimed in claim 1, characterized in that the active compound is the cannabinoid receptor (CB 1) antagonist SR 141716.
